(19) United States
(12) Patent Application Publication
(10) Pub. No.: US 2025/0082873 A1
(43) Pub. Date: Mar. 13, 2025

(54) Inhaler Device

(71) Applicant: Sandra Garland, Paducah, KY (US)

(72) Inventor: Sandra Garland, Paducah, KY (US)

(21) Appl. No.: 18/415,841

(22) Filed: Jan. 18, 2024

Related U.S. Application Data
(60) Provisional application No. 63/581,059, filed on Sep. 7, 2023.

(51) Int. Cl. A61M 15/00; A61M 15/06

(52) U.S. Cl. CPC A61M 15/0028; A61M 15/0025; A61M 15/0068; A61M 15/06

(57) ABSTRACT

A compact inhaler device is disclosed that is a miniature, more ergonomic design for medical inhalers. The design is more convenient and allows an owner to place the inhaler in their pocket, bag, or purse without taking up much space. The compact inhaler device comprises a body component that is configured in a rectangular shape that resembles a vape. Thus, the body component is inline versus an L-shaped inhaler. The device comprises a refillable medication cartridge component that abuts a piston to convert the liquid inside the cartridge to a vaporized state. Accordingly, the device works and looks like a vape but is for medical purposes only.

### CROSS-REFERENCE TO RELATED APPLICATION

The present application claims priority to, and the benefit of, U.S. Provisional Application No. 63/581,059, which was filed on Sep. 7, 2023, and is incorporated herein by reference in its entirety.

### FIELD OF THE INVENTION

The present invention relates generally to the field of compact inhaler devices. More specifically, the present invention relates to a medical inhaler designed as a more convenient, lightweight, and smaller option compared to large bulky inhalers. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in compact inhaler devices. Generally, people who require inhalers may have trouble storing and transporting them due to their bulky sizes. Specifically, the inhaler is too large to fit in a pocket and can cause discomfort when walking or sitting down. Further, putting the inhaler in a purse or backpack can take up considerable space. A smaller, more compact inhaler would be useful.

Generally, an inhaler is a device used for delivering medication via inhalation. Inhalers typically use a compressed-gas propellant to deliver a metered dose of medicine. When the inhaler is activated, a fixed amount of the medicine is suspended in the propellant and is expelled from the mouthpiece of the inhaler. A need existed in the art for a compact inhaler. A compact implementation of an inhaler would allow for the device to be carried in a user's pocket, in a small purse, or worn on the body thereby making it available to a patient or user for medical emergencies or simple convenience.

Currently, many patients with ailments are provided an inhaler to provide dosages of medication. For example, an asthma patient may be provided a stimulant to assist mucus and reducing inflammation in clearing up breathing passages. Thus, when the patient experiences asthma exacerbations or as a daily maintenance medication to control symptoms, the patient can put the inhaler in front of their mouth and activate the inhaler spray, delivering a puff or puffs of the drug into the lungs in order to relieve the symptoms.

A known pressurized metered dose inhaler (pMDI) inhaler, for the inhalation of drugs, has an actuator housing at least partially defining a flow passageway extending through the inhaler from an air inlet to an outlet. A pressurized metered dose canister is held by the actuator. The canister includes a valve stem and a metering valve arranged to sit in a valve stem block formed on the housing and a main canister body of the canister may be moved relative to the housing and valve stem so as to operate the metering valve and fire a metered puff of propellant and active drug through the valve stem block and into the flow passageway. By depressing the canister, when a user inhales through a mouthpiece of the housing, air may be drawn into the housing between the canister and an inner wall of the housing, and may flow along past the canister towards the outlet.

In most known inhalers, the canister is placed in a holder but the mouthpiece remains accessible to catch debris between uses. When inhalers are not cleaned regularly or properly, the mouthpieces may be clogged or accumulate debris. Further, the inhalers are bulky and difficult to carry in a pocket or small purse.

Accordingly, there is a need for a compact inhaler device that is a more convenient, lightweight, and smaller option compared to large bulky inhalers. There is also a need for a compact inhaler that may be carried conveniently by a user.

Therefore, there exists a long-felt need in the art for a compact inhaler device that provides users with a medical inhaler designed as a more convenient, lightweight, and smaller option compared to large bulky inhalers. There is also a long-felt need in the art for a compact inhaler device that allows users to easily place the inhaler in a purse, pocket, or backpack without taking up much space. Further, there is a long-felt need in the art for a compact inhaler device that functions like a vape when medication is placed within the inhaler to properly distribute the medication as needed. Moreover, there is a long-felt need in the art for a device that offers significant convenience for anyone who needs to use an inhaler. Further, there is a long-felt need in the art for a compact inhaler device that can be refillable or disposable. Finally, there is a long-felt need in the art for a compact inhaler device that looks like a vape but is for medical purposes only.

The subject matter disclosed and claimed herein, in one embodiment thereof, comprises a compact inhaler device. The device is a miniature, more ergonomic design for medical inhalers. The design is more convenient and allows an owner to place the inhaler in their pocket, bag, or purse without taking up much space. The compact inhaler device comprises a body component that is configured in a rectangular shape that resembles a vape. Thus, the body component is inline versus an L-shaped inhaler. The device comprises a refillable medication cartridge component that abuts a piston to convert the liquid inside the cartridge to a vaporized state. Accordingly, the device works and looks like a vape but is for medical purposes only.

In this manner, the compact inhaler device of the present invention accomplishes all of the foregoing objectives and provides users with a device that provides users with a compact inhaler. The device is a vape-like device that is for medical purposes only. The device can be manufactured of a disposable, plastic material.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a compact inhaler device. The device is a miniature, more ergonomic design for medical inhalers. The design is more convenient and allows an owner to place the inhaler in their pocket, bag, or purse without taking up much space. The compact inhaler device comprises a body component that is configured in a rectangular shape that resembles a vape. The device comprises a single dose or refillable medication cartridge component that abuts a piston to convert the liquid inside the cartridge to a vaporized state. Accordingly, the device works and looks like a vape but is for medical purposes only.

In one embodiment, the compact inhaler device comprises a body component in a rectangular shape that resembles a vape. Typically, the body component is an inline shape versus an L-shape, as most prior art inhalers are. In one embodiment, the body component is an elongated rectangular cylinder made of plastic or alloy stainless steel, carbon steel, iron, copper aluminum, or other suitable material and has approximate dimensions of 27×13×5 millimeters to 30×17×8 millimeters. The body component has an open end and a mouthpiece, wherein a removable medication cartridge component is adapted to seat within the open end of the body component.

In one embodiment, the body component comprises a mouthpiece, an open end, and an activation button aperture. The open end is operable to insert a medication cartridge component. A medication cartridge component may be inserted in the open end of the body component to dispense medicine from the mouthpiece when actuated. Further, an activation button is moveable in the button aperture. Pressing the activation button pushes an inserted medication cartridge component out of the body component. Inhaling on the mouthpiece releases medicine from the medication cartridge component into a user's mouth.

In one embodiment, the body component and the mouthpiece are molded from plastic or similar materials, such as ABS, polycarbonate, PETE, acrylic, or polystyrene. The mouthpiece is cylindrically shaped and is of sufficient diameter to release medication through the mouthpiece to a user's mouth to be inhaled by a user, during use. The mouthpiece extends perpendicularly from the body component and allows access to the interior of the body component.

In one embodiment the medication cartridge component comprises a rescue medicament for a respiratory ailment. Another implementation is where the medication cartridge component contains a single dose of the medicament. For example, rescue medications such as Beclomethasone, fluticasone, ciclesonide, mometasone, budesonide, flunisolide, and albuterol sulfate may be provided for asthma. Although an amount of medicament sufficient for a single use is contained in the medication cartridge component in this example, an amount of medicament allowing for multiple uses may be carried by the medication cartridge component, if the inhaler device is designed to be used for more than one use.

In one embodiment, the medication cartridge component has a generally cylindrically shaped container that holds the medicament. The interior volume of the container is sealed off to contain the medicament. The container has a cylindrical wall that tapers in to an annular collar. The annular collar has a closed end that has a spray stem extending into the container. One end of the spray stem is attached to an internal piston in the container or in the body component. Pushing the stem into the container pressurizes the medicament that is propelled to be emitted through the stem when the stem is released. Thus, the liquid inside the cartridge is converted to a vaporized state for use.

In another embodiment, the medication cartridge component comprises a propellant gas, such that the cartridge is under pressure. The gas can be nitrogen, argon, carbon dioxide, etc. The medication cartridge component comprises a valve at one end. Thus, once inserted into the body component, the valve abuts a piston assembly within the body component. The piston assembly pushes against the valve to release the medicant into the mouthpiece in a vaporized state.

In one embodiment, the medication cartridge component is held in place in the open end via friction fit or by contact with a small ridge in the body component providing a friction contact point.

In one embodiment, the body component comprises a cover that fits over the mouthpiece, protecting the mouthpiece from dirt, debris and germs when not in use. A user merely grasps the cover and pulls to remove the cover for use and places it back over the mouthpiece for storage.

In one embodiment, the body component comprises a gauge implemented as a transparent window of plastic or glass that shows the level of inhalant remaining in the medication cartridge component thereof.

In one embodiment, the body component comprises a keyhole cut through the side of the body component to allow the inhaler device to be attached to other objects, such as a key ring. In one embodiment, the key ring has an end that forms a circle. One end may be inserted through the keyhole to attach the inhaler device to the key ring along with other objects, such as keys or a fob. The user may thus carry the compact inhaler device in a pocket or any other convenient carrying place. The user may therefore have ready access to the inhaler device should there be a need for a medicament dosage. The compact inhaler device may be for a single use where the cartridge is packaged with the compact inhaler device. Alternatively, the device may be used with a refill cartridge for multi-use functionality. Thus, after a use, the user may discard the device or replace the empty cartridge. The user may then insert a fresh cartridge into the body component, as needed, and reuse the compact inhaler device.

In one embodiment, an adherence/event detection module may be embedded or attached to the compact inhaler device. The adherence/event detection module allows detection of the use of the compact inhaler device and may allow recording the use, as well as transmitting data to an external device or system for purposes such as compliance, treatment, or diagnosis in relation to respiratory ailments.

In one embodiment, the compact inhaler device provides a medical inhaler that may be easily accessible to provide a puff of medicament such as a rescue medicament for a respiratory ailment. The compact inhaler device may be carried by a user for extended periods of time before use. The compact inhaler device has a storage configuration that stores the medication cartridge component within the body component. When a user desires to use the compact inhaler device, the user removes the cap from the mouthpiece. The cap may then be removed to access the mouthpiece. A user puts their mouth on the mouthpiece and inhales to release the medicament through the now accessible mouthpiece.

In operation, to dispense a puff, the user holds the inhaler device and puts the mouthpiece in their mouth. The user may then push the medication cartridge component downward in the body component. When the cartridge is pushed down, it contacts a piston assembly to cause pressure to force the stored medicament out of the cartridge and through the mouthpiece, thus, delivering a metered puff of medicament. The medicament is forced out through the open end of the mouthpiece and into the user's mouth in a vaporized state. Ideally, a user will push down on the cartridge at the same time the user inhales, thus maximizing the inhalation of the medicament dose. The cartridge ideally contains sufficient medicament for multiple puffs to address an emergency event.

In yet another embodiment, the compact inhaler device comprises a plurality of indicia.

In yet another embodiment, a method of utilizing a more compact inhaler is disclosed. The method includes the steps of providing a compact inhaler device comprising a body component and medication cartridge component. The method also comprises inserting a medication cartridge component into the body component. Further, the method comprises utilizing a nozzle of the body component to inhale vaporized medicine from the medication cartridge component. The method also comprises removing and replacing the medication cartridge component, as needed. Finally, the method comprises disposing of the device and purchasing a new device, as needed.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the compact inhaler device of the present invention in accordance with the disclosed architecture;

FIGS. 2A-B illustrate a perspective view of one embodiment of the compact inhaler device of the present invention showing the device components in accordance with the disclosed architecture;

FIGS. 3A-B illustrate a perspective view of one embodiment of the compact inhaler device of the present invention showing how the medication cartridge component is inserted into the device in accordance with the disclosed architecture;

FIGS. 4A-B illustrate a perspective view of one embodiment of the compact inhaler device of the present invention showing how the medication cartridge component abuts the piston assembly in accordance with the disclosed architecture;

FIG. 5 illustrates a perspective view of one embodiment of the compact inhaler device of the present invention stored in a backpack in accordance with the disclosed architecture; and

FIG. 6 illustrates a flowchart showing the method of utilizing a more compact inhaler in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a compact inhaler device that provides users with a medical inhaler designed as a more convenient, lightweight, and smaller option compared to large bulky inhalers. There is also a long-felt need in the art for a compact inhaler device that allows users to easily place the inhaler in a purse, pocket, or backpack without taking up much space. Further, there is a long-felt need in the art for a compact inhaler device that functions like a vape when medication is placed within the inhaler to properly distribute the medication as needed. Moreover, there is a long-felt need in the art for a device that offers significant convenience for anyone who needs to use an inhaler. Further, there is a long-felt need in the art for a compact inhaler device that can be refillable or disposable. Finally, there is a long-felt need in the art for a compact inhaler device that looks like a vape but is for medical purposes only.

The present invention, in one exemplary embodiment, is a novel compact inhaler device. The device is a miniature, more ergonomic design for medical inhalers. The design is more convenient and allows an owner to place the inhaler in their pocket, bag, or purse without taking up much space. The compact inhaler device comprises a body component that is configured in a rectangular shape that resembles a vape. Thus, the body component is inline versus an L-shaped inhaler. The device comprises a refillable medication cartridge component that abuts a piston to convert the liquid inside the cartridge to a vaporized state. Accordingly, the device works and looks like a vape but is for medical purposes only. The present invention also includes a novel method of utilizing a more compact inhaler. The method includes the steps of providing a compact inhaler device comprising a body component and medication cartridge component. The method also comprises inserting a medication cartridge component into the body component. Further, the method comprises utilizing a nozzle of the body component to inhale vaporized medicine from the medication cartridge component. The method also comprises removing and replacing the medication cartridge component, as needed. Finally, the method comprises disposing of the device and purchasing a new device, as needed.

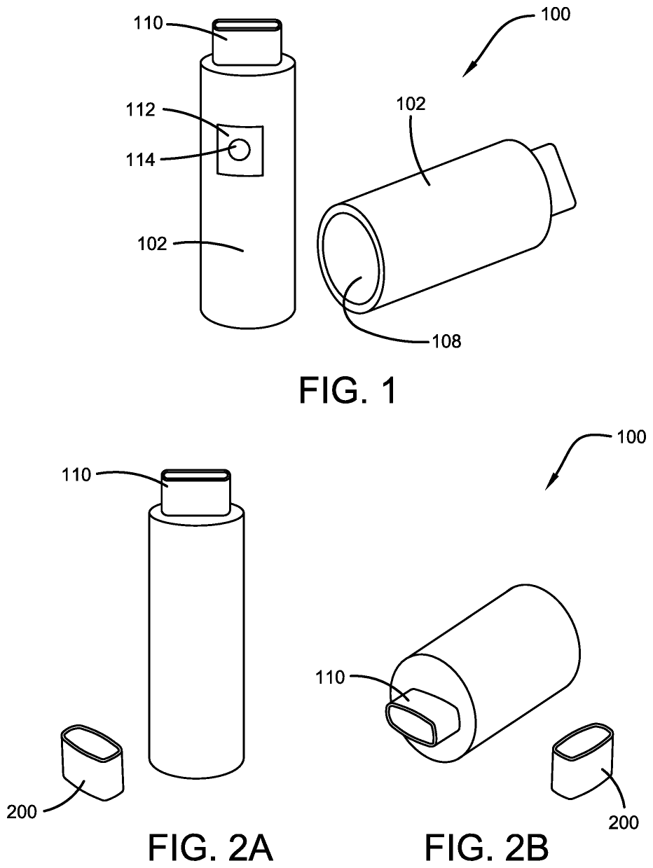

Referring initially to the drawings, FIG. 1 illustrates a perspective view of one embodiment of the compact inhaler device 100 of the present invention. In the present embodiment, the compact inhaler device 100 is an improved compact inhaler device 100 that provides a user with a medical inhaler designed as a more convenient, lightweight, and smaller option compared to large bulky inhalers. The design is more convenient and allows a user to place the inhaler device 100 in their pocket, bag 502, or purse without taking up much space. Specifically, the compact inhaler device 100 comprises a body component 102 with a medication cartridge component 104. The device 100 comprises a single dose or refillable medication cartridge component 104 that abuts a piston assembly 106 to convert the liquid inside the cartridge 104 to a vaporized state. Accordingly, the device 100 works and looks like a vape but is for medical purposes only.

Generally, the compact inhaler device 100 comprises a body component 102 in a rectangular, cylindrical, or oval shape, etc., or any other suitable shape as is known in the art. Further, the body component 102 is an inline shape versus an L-shape (as most prior art inhalers are) and thus resembles a vape. In one embodiment, the body component is an elongated cylinder made of plastic or alloy stainless steel, carbon steel, iron, copper aluminum, or other suitable material and has approximate dimensions of 27×13×5 millimeters to 30×17×8 millimeters. The body component can be any suitable shape and size as is known in the art, depending on the wants and/or needs of a user. Typically, the body component 102 has an open end 108 and a mouthpiece 110, wherein a removable medication cartridge component 104 is adapted to seat within the open end 108 of the body component 102.

As shown in FIGS. 2A-B, the body component 102 comprises a mouthpiece 110, an open end 108, and an activation button aperture 112. The open end 108 is operable to insert a medication cartridge component 104. A medication cartridge component 104 may be inserted in the open end 108 of the body component 102 to dispense medicine from the mouthpiece 110 when actuated. Further, an activation button 114 is moveable in the button aperture 112. Pressing the activation button 114 pushes an inserted medication cartridge component 104 out of the body component 102 when empty. Further, inhaling on the mouthpiece 110, releases medicine from the medication cartridge 104 component into a user's mouth.

Generally, the body component 102 and the mouthpiece 110 are molded from metal, plastic, or similar materials, such as stainless steel, ABS, polycarbonate, PETE, acrylic, or polystyrene, etc. The mouthpiece 110 is cylindrically shaped and is of sufficient diameter to release medicament through the mouthpiece 110 to a user's mouth to be inhaled by a user, during use. The mouthpiece 110 extends perpendicularly from the body component 102 and allows access to the interior of the body component 102.

Further, the body component 102 comprises a cover 200 that fits over the mouthpiece 110, protecting the mouthpiece 110 from dirt, debris, and germs when not in use. A user merely grasps the cover 200 and pulls to remove the cover 200 for use and places it back over the mouthpiece 110 for storage. The cover 200 can be any suitable shape and size as is known in the art, as long as it acts to cover the mouthpiece 110 and protect it when the device 100 is not in use.

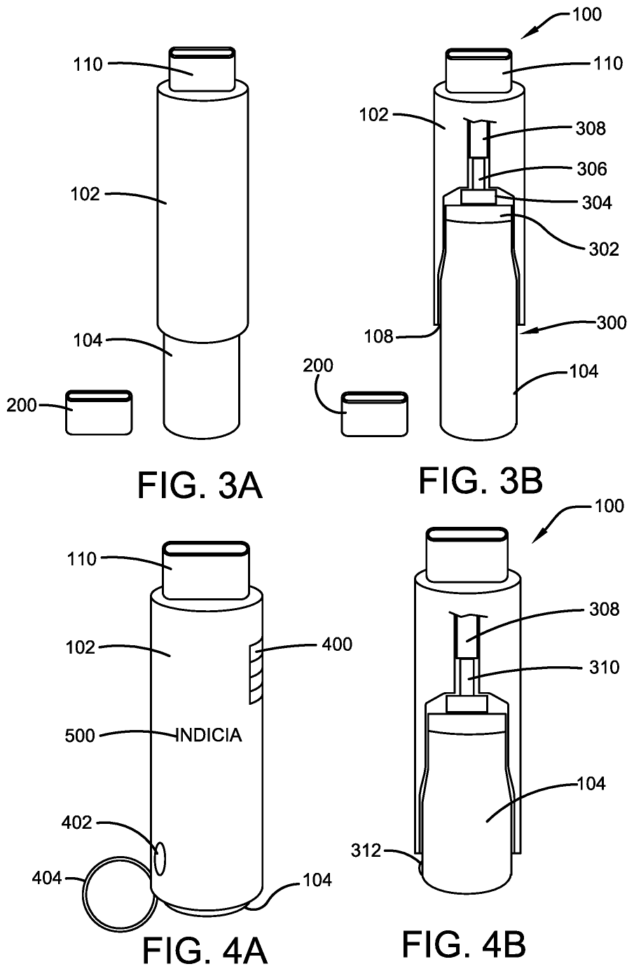

As shown in FIGS. 3A-B, the medication cartridge component 104 comprises a rescue medicament for a respiratory ailment. Another implementation is where the medication cartridge component 104 contains a single dose of the medicament. For example, rescue medications such as Beclomethasone, fluticasone, ciclesonide, mometasone, budesonide, flunisolide, albuterol sulfate, etc., may be provided for asthma. Although an amount of medicament sufficient for a single use is contained in the medication cartridge component 104 in this example, an amount of medicament allowing for multiple uses may be carried by the medication cartridge component 104, if the inhaler device 100 is designed to be used for more than one use.

Further, the medication cartridge component 104 is a generally cylindrically shaped container that holds the medicament. The interior volume of the cartridge 104 is sealed off to contain the medicament. In one embodiment, the cartridge 104 has a cylindrical wall 300 that tapers into an annular collar 302. The annular collar 302 has a closed-end 304 that has a spray stem 306 extending into the cartridge 104. One end of the spray stem 306 is attached to an internal piston assembly 308 in the body component 102. Pushing the stem 306 into the cartridge 104 pressurizes the medicament that is propelled to be emitted through the stem 306 when the stem 306 is released. Thus, the liquid inside the cartridge 104 is converted to a vaporized state for use.

In another embodiment, the medication cartridge component 104 comprises a propellant gas, such that the cartridge 104 is under pressure. The gas can be nitrogen, argon, carbon dioxide, etc. The medication cartridge component 104 comprises a valve 310 at one end. Thus, once inserted into the body component 102, the valve 310 abuts a piston assembly 308 within the body component 102. The piston assembly 308 pushes against the valve 310 to release the medicant into the mouthpiece 110 in a vaporized state.

Generally, the medication cartridge component 104 is held in place in the open end 108 via friction fit or by contact with a small ridge 312 in the body component 102 providing a friction contact point, or any other suitable securing means as is known in the art.

As shown in FIGS. 4A-B and 5, in one embodiment, the body component 102 comprises a gauge 400 implemented as a transparent window of plastic or glass that shows the level of inhalant remaining in the medication cartridge component 104 thereof. The gauge 400 can be positioned in any suitable place on the body component 102 as is known in the art.

In another embodiment, the body component 102 comprises a keyhole 402 cut through the side of the body component 102 to allow the inhaler device 100 to be attached to other objects, such as a key ring 404. In this embodiment, the key ring 404 has an end that forms a circle. One end may be inserted through the keyhole 402 to attach the inhaler device 100 to the key ring 404 along with other objects, such as keys or a fob. The user may thus carry the compact inhaler device 100 in a pocket or any other convenient carrying place. The user may therefore have ready access to the inhaler device 100 should there be a need for a medicament dosage. The compact inhaler device 100 may be for a single use where the cartridge 104 is packaged with the compact inhaler device 100. Alternatively, the device 100 may be used with a refill cartridge 104 for multi-use functionality. Thus, after a use, the user may discard the device 100 or replace the empty cartridge 104. The user may then insert a fresh cartridge 104 into the body component 102, as needed, and reuse the compact inhaler device 100.

In another embodiment, an adherence/event detection module (not shown) may be embedded or attached to the compact inhaler device 100. The adherence/event detection module allows detection of the use of the compact inhaler device 100 and may allow recording the use, as well as transmitting data to an external device or system for purposes such as compliance, treatment, or diagnosis in relation to respiratory ailments.

Generally, the compact inhaler device 100 provides a medical inhaler that may be easily accessible to provide a puff of medicament such as a rescue medicament for a respiratory ailment. The compact inhaler device 100 may be carried by a user for extended periods of time before use. The compact inhaler device 100 has a storage configuration that stores the medication cartridge component 104 within the body component 102. When a user desires to use the compact inhaler device 100, the user removes the cover 200 from the mouthpiece 110. The cover 200 may then be removed to access the mouthpiece 110. A user puts their mouth on the mouthpiece 110 and inhales to release the medicament through the now accessible mouthpiece 110.

In operation, to dispense a puff, the user holds the inhaler device 100 and puts the mouthpiece 110 in their mouth. The user may then push the medication cartridge component 104 downward in the body component 102. When the cartridge 104 is pushed down, it contacts a piston assembly 308 to cause pressure to force the stored medicament out of the cartridge 104 and through the mouthpiece 110, thus, delivering a metered puff of medicament. The medicament is forced out through the open end of the mouthpiece 110 and into the user's mouth in a vaporized state. Ideally, a user will push down on the cartridge 104 at the same time the user inhales, thus maximizing the inhalation of the medicament dose. The cartridge 104 ideally contains sufficient medicament for multiple puffs to address an emergency event.

In yet another embodiment, the compact inhaler device 100 comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as, but not limited to, patterns, logos, emblems, images, symbols, designs, letters, words, characters, animals, advertisements, brands, etc., that may or may not be inhaler, medicine, or brand related.

FIG. 6 illustrates a flowchart of the method of utilizing a more compact inhaler. The method includes the steps of at 600, providing a compact inhaler device comprising a body component and medication cartridge component. The method also comprises at 602, inserting a medication cartridge component into the body component. Further, the method comprises at 604, utilizing a nozzle of the body component to inhale vaporized medicine from the medication cartridge component. The method also comprises at 606, removing and replacing the medication cartridge component, as needed. Finally, the method comprises at 608, disposing of the device and purchasing a new device, as needed.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “compact inhaler device”, “inhaler device”, “compact device”, and “device” are interchangeable and refer to the compact inhaler device 100 of the present invention.

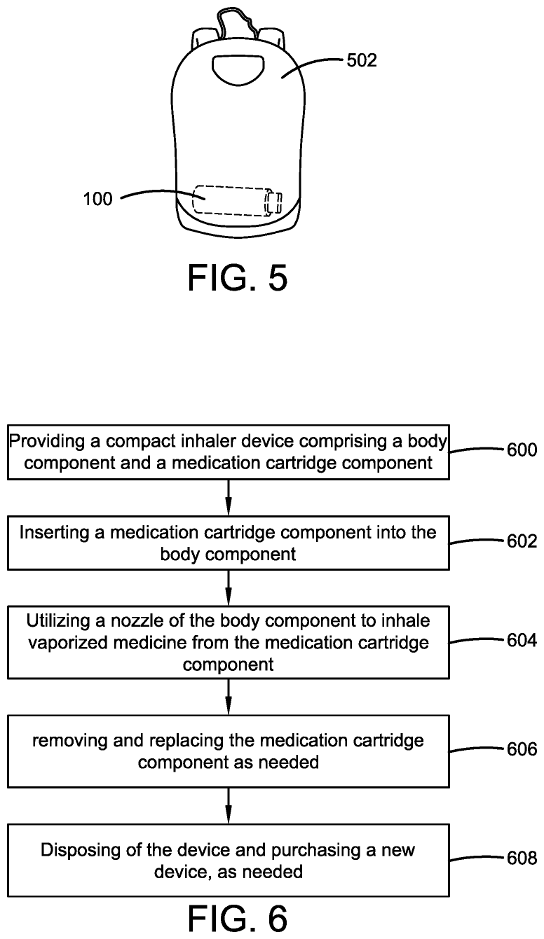

Notwithstanding the foregoing, the compact inhaler device 100 of the present invention can be of any suitable size and configuration as is known in the art without affecting the overall concept of the invention, provided that it accomplishes the above-stated objectives. One of ordinary skill in the art will appreciate that the compact inhaler device 100 as shown in FIGS. 1-6 is for illustrative purposes only, and that many other sizes and shapes of the compact inhaler device 100 are well within the scope of the present disclosure. Although the dimensions of the compact inhaler device 100 are important design parameters for user convenience, the compact inhaler device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A compact inhaler device comprising:
a body component; and
a medication cartridge component;
wherein the medication cartridge component is inserted into the body component;
wherein the medication cartridge component abuts a piston assembly within the body component to convert liquid medication from inside the medication cartridge component to be expelled in a vaporized state; and
further wherein inhaling on the body component releases medicine from the medication cartridge component into a user's mouth.

2. The compact inhaler device of claim 1, wherein the body component is configured in a rectangular, cylindrical, or oval shape.

3. The compact inhaler device of claim 2, wherein the body component is an inline shape and not an L-shape.

4. The compact inhaler device of claim 3, wherein the body component comprises an open end and a mouthpiece at an opposing end.

5. The compact inhaler device of claim 4, wherein the medication cartridge component is adapted to be inserted within the open end of the body component.

6. The compact inhaler device of claim 5, wherein the body component comprises an activation button aperture with an activation button.

7. The compact inhaler device of claim 6, wherein the activation button is moveable in the button aperture and pressing the activation button pushes an inserted medication cartridge component out of the body component when empty.

8. The compact inhaler device of claim 7, wherein the mouthpiece is cylindrically shaped and extends perpendicularly from the body component to release medicament through the mouthpiece to a user's mouth to be inhaled by a user, during use.

9. The compact inhaler device of claim 8, wherein the body component comprises a cover that fits over the mouthpiece, protecting the mouthpiece from dirt, debris and germs when not in use.

10. The compact inhaler device of claim 9, wherein the medication cartridge component comprises a rescue medicament for a respiratory ailment.

11. The compact inhaler device of claim 10, wherein the compact inhaler device is a single use device.

12. The compact inhaler device of claim 10, wherein the compact inhaler device is a multi-use device.

13. The compact inhaler device of claim 10, wherein the medication cartridge component is a generally cylindrically shaped container that holds the medicament and an interior volume of the medication cartridge component is sealed off to contain the medicament.

14. The compact inhaler device of claim 13, wherein the medication cartridge component has a cylindrical wall that tapers into an annular collar, that has a spray stem extending and which abuts an internal piston assembly in the body component, pressurizing the medicament that is propelled to be emitted through the mouthpiece.

15. The compact inhaler device of claim 10, wherein the medication cartridge component comprises a propellant gas and has a valve at one end that abuts a piston assembly within the body component, the piston assembly pushes against the valve to release the medicant into the mouthpiece in a vaporized state.

16. A compact inhaler device comprising:
a body component comprising an open end and a mouthpiece at an opposing end; and
a medication cartridge component comprising a rescue medicament for a respiratory ailment;
wherein the medication cartridge component is inserted into the open end of the body component;
wherein the medication cartridge component is held in place in the open end via friction fit;
wherein the body component comprises an activation button aperture with an activation button;
wherein the activation button is moveable in the button aperture and pressing the activation button pushes an inserted medication cartridge component out of the body component when empty;
wherein the body component comprises a cover that fits over the mouthpiece, protecting the mouthpiece from dirt, debris and germs when not in use;
wherein the medication cartridge component comprises a propellant gas and has a valve at one end that abuts a piston assembly within the body component;
wherein the piston assembly pushes against the valve to release the rescue medicant into the mouthpiece in a vaporized state; and
further wherein inhaling on the body component releases vaporized rescue medicant from the medication cartridge component into a user's mouth.

17. The compact inhaler device of claim 16, wherein the body component comprises a gauge for showing level of inhalant remaining in the medication cartridge component.

18. The compact inhaler device of claim 16, wherein the body component comprises a keyhole cut through a side of the body component to allow the compact inhaler device to be attached to other objects, such as a key ring.

19. The compact inhaler device of claim 16 further comprising a plurality of indicia.

20. A method of utilizing a more compact inhaler, the method comprising the following steps:
providing a compact inhaler device comprising a body component and medication cartridge component;
inserting a medication cartridge component into the body component;
utilizing a nozzle of the body component to inhale vaporized medicine from the medication cartridge component;
removing and replacing the medication cartridge component, as needed; and
disposing of the device and purchasing a new device, as needed.
